(19) United States
(12) Patent Application Publication
(10) Pub. No.: US 2023/0294146 A1
(43) Pub. Date: Sep. 21, 2023

(54) METHOD AND INSTALLATION FOR CLEANING A FILTER MATERIAL

(71) Applicants: UNIVERSITE DE BORDEAUX, Bourdeaux (FR); CENTRE NATIONAL DE LA RECHERCHE SCIENTIFIQUE, Paris, Cedex 16 (FR); INSTITUT POLYTECHNIQUE DE BORDEAUX, Telence cedex (FR)

(72) Inventors: Cyril AYMONIER, Begles (FR); Guillaume AUBERT, Talence (FR); Anais CARIO, Begles (FR)

(21) Appl. No.: 18/017,048

(22) PCT Filed: Jul. 20, 2022
(86) PCT No.: PCT/EP2021/070214

§ 371 Date: Jan. 19, 2023

(30) Foreign Application Priority Data
Jul. 21, 2020 (FR) FR2007648

(51) Int. Cl. B08B 7/00; A41D 13/11; A61L 2/28

(52) U.S. Cl. CPC B08B 7/0021; A41D 13/1138; A61L 2/28; B01D 39/1623

(57) ABSTRACT

A method for cleaning at least one filter material, in particular a filter material used in the production of a respiratory protection mask (M), comprising the step of subjecting this filter material to a supercritical fluid, preferably supercritical CO₂, with a density of less than 0.3 g/ml.

### TECHNICAL FIELD

The present invention concerns cleaning a filter material and more particularly but not exclusively cleaning respiratory protection masks with a view to their reuse.

### PRIOR ART

The COVID 19 pandemic revealed the need to be able to reuse respiratory protection masks, in particular those of FFP2 or FFP3 type, to confront the problem of supplying these masks.

These masks are made using a filter material based on a non-woven fabric known as meltblown fabric, the particular feature of which is to be charged electrostatically. It is the presence of surface electrical charges that enable the fibers of the material to capture the smallest particles, in particular viruses. The meltblown type non-woven fabric used for the production of masks generally based on polypropylene fibers, in particular because of the highly tribo-electric character of this material, that is to say its high capacity to attract electric charges at its surface and thus to form an electrostatic barrier.

The cleaning of contaminated masks must therefore preserve the filter material mechanically, exert a biocidal effect, remove the traces of any soiling, and affect as little as possible the electrostatic properties of the filter material, so as not to degrade its capacity to retain the smallest particles.

Moreover, cleaning must not cause any attachments or seals made of foam fixed to the mask by gluing them thereto to come unstuck.

Finally, cleaning must be employable at low cost, in a non-polluting manner and on a large scale.

It has been proposed in the prior art to use supercritical carbon dioxide CO₂ to clean textiles. Supercritical CO₂ is used under conditions conferring on it a high density, the solubility of pollutants increasing with the density of the supercritical CO₂, as taught in the publication by Aymonier, Cyril, et al. “Materials Processing and Recycling with Near-and Supercritical CO₂-based Solvents”, Supercritical and Other High-pressure Solvent Systems. 2018. 304-339.

There is also in the publication by Perrut, Michel “Sterilization and virus inactivation by supercritical fluids (a review)”, The Journal of Supercritical Fluids 66 (2012): 359-371, a reminder that it is known to use supercritical CO₂ to sterilize very diverse products at pressures varying according to the product, sometime with addition of an oxidant such as hydrogen peroxide or peracetic acid.

The first tests carried out in-house by the inventors showed that the use of supercritical CO₂ under the above conditions as disclosed by the prior art caused deterioration of the filtration properties of FFP2 masks because in particular of damage to their fibrous structure and loss of their electrostatic properties.

### STATEMENT OF INVENTION

The invention consequently aims to find a new method that is relatively simple and economical to use for cleaning masks, in particular of FFP2 type, in such a manner as to enable them to be reused at least twice.

### SUMMARY OF THE INVENTION

Thus the invention has for object a method for cleaning a filter material, in particular a filter material used in the production of a respiratory protection mask, including the step consisting in subjecting that filter material to a supercritical fluid, preferably supercritical CO₂, with a mass per unit volume less than 0.3 g/mL. This supercritical fluid is preferably used when mixed with at least one additional compound, in particular a biocidal compound, and/or an organic polar co-solvent, as described in detail hereinafter. Alternatively, the supercritical fluid is used pure.

Surprisingly, whereas the use of supercritical CO₂ with a mass per unit volume greater than or equal to 0.3 g/mL very significantly degrades the filtration properties of the filter material for fine particles, the use of supercritical CO₂ with a lower mass per unit volume enables it to be cleaned without unduly affecting its electrostatic properties, to eliminate traces of soiling, if any, and to exert a biocidal action when mixed with one or more other compounds having a biocidal effect, as described in detail below.

The supercritical fluid used, and in particular the supercritical CO₂ used, preferably has a mass per unit volume less than or equal to 0.25 g/mL, better still less than or equal to 0.2 g/mL, even better less than or equal to 0.19, 0.18, 0.17 or 0.16 g/mL, the mass per unit volume preferably being between 0.14 and 0.16 g/mL inclusive, being even more preferably equal to 0.15 g/mL.

The temperature of the supercritical fluid used, in particular of the supercritical CO₂, is preferably less than or equal to 130° C., in particular greater than the critical temperature of the CO₂ and less than or equal to 130° C., better between 50° C. and 130° C. inclusive, even better between 70° C. and 100° C. inclusive.

The pressure of the supercritical fluid used, in particular of the supercritical CO₂, is preferably less than or equal to 100 bar, in particular greater than the critical pressure of the CO₂ and less than or equal to 100 bar, preferably between 75 and 100 bar inclusive.

The filter material is preferably exposed to a mixture of the supercritical fluid, in particular supercritical CO₂, and at least one additional biocidal compound.

The additional biocidal compound or compounds is/are preferably present in a total molar content of additional biocidal compounds less than or equal to 2%, in particular 1.5%, relative to the total number of moles of the mixture, better 1.25%, even better 1.1%, or even 1%, 0.9%, 0.8%, 0.7%, 0.6% or 0.5%.

The mixture may include at least 0.05% total molar fraction of biocidal compound(s), in particular H₂O₂, better at least 0.1%, even better at least 0.2%, 0.3%, 0.5% or 1%.

When the biocidal compound is H₂O₂ in 30 wt % solution in water the mixture preferably includes between 1 and 2 mole % biocidal compound(s), better between 1.3 and 1.7 mole %, for example approximately 1.4 mole %.

The additional biocidal compound(s) may be chosen from compounds generating free radicals, in particular oxidants such as hydrogen peroxide H₂O₂, acids, in particular carboxylic acids, for example acetic acid, and mixtures thereof.

In particular when the supercritical fluid is supercritical CO₂ and the biocidal compound(s) is/are in aqueous solution, given that water has poor solubility in supercritical CO₂, an organic co-solvent having an affinity with water and CO₂ may be added to the mixture to enable the water to dissolve in the supercritical CO₂.

The co-solvent may be chosen from alcohols with three carbon atoms or fewer, preferably ethanol, acids, in particular carboxylic acids, with three carbon atoms or fewer, preferably acetic acid, polyether solutions, preferably of polyethylene glycol (PEG) having in particular a molecular mass less than 200 g·mol⁻¹, acetone, dimethylsulfoxide (DMSO), and mixtures thereof.

The co-solvent may moreover be added to the mixture to enable the polarity of the CO₂ or other supercritical fluid to be increased in such a manner as to improve its capacity for stabilization of pollutants and soiling and therefore for cleaning.

The total molar content of organic solvent(s) may be less than or equal to 1.5% relative to the total number of moles of the mixture, better less than or equal to 1.25%, better still 1.1%, 1% or 0.9%, for example approximately 0.8 mole % of ethanol.

The total content of organic co-solvent(s) may be at least 0.1 mole % relative to the total number of moles of the mixture, better at least 0.3%, even better at least 0.5%.

The filter material is preferably exposed to the supercritical fluid, in particular to the supercritical CO₂, without mechanical agitation produced by stirring, by ultrasound or by imparting movement to the filter material.

The inventors surprisingly found that controlled expansion made it possible to create a movement drawing soiling out of the filter material without this damaging the fibrous structure because of the departure of the supercritical fluid that has diffused into the latter. The rate of expansion is therefore advantageously between 10 bar/min and 90 bar/min inclusive, better between 20 bar/min and 80 bar/min inclusive, even better between 60 bar/min and 80 bar/min inclusive, preferably being of the order of 70 bar/min. The expansion may be effected with at least one decompression stage, for example at a value P₀/2, where P₀ designates the maximal supercritical fluid pressure during treatment. The expansion may therefore correspond to a pressure variation between 25 and 50 bar inclusive between each stage. The expansion may be automated, for example by means of a back pressure regulator (BPR) system.

The supercritical fluid is preferably recycled after its expansion in order to be reused.

The additional compound(s), such as the aforementioned biocidal compound(s), may be recovered by a liquid/gas separator once the supercritical fluid has reverted to the gaseous state.

The gaseous supercritical fluid may be fed into a filter before being stored for reuse.

The treatment may be carried out by disposing the filter material, for example the masks, in an enclosure and then using a pump to inject the supercritical fluid alone or mixed with one or more additional compounds. The latter may be introduced into the treatment enclosure before the supercritical fluid. It is furthermore possible to inject the other additional compound or compounds into the enclosure using a dedicated pump.

The duration of exposure of the filter material to the supercritical fluid is sufficient to enable the required effects to be obtained; this duration is preferably between one minute and two hours inclusive, being for example of the order of one hour.

The method according to the invention may include a step of counting the number of cleaning cycles to which the filter material has been subjected, in particular if at the end of a given number of cleaning cycles the filter properties cease to conform.

To facilitate this monitoring the filter material or mask may carry a visual indicator the color of which fades as the cleaning cycles proceed. In this case the method may include a step of verifying that the indicator is still colored to more than a certain degree before permitting reuse of the filter material.

The filter material or the mask may moreover feature an identifier, for example a bar code or a QR code, and the method may include the step consisting in storing in a database information as to the number of cleaning cycles to which the filter material or the mask has been subjected. The method may include updating this base before and/or after the cleaning operation and verifying that the number of cleaning cycles remains below a given value, guaranteeing that the mask is conform.

From a predefined number of cleaning cycles the cleaning method may where appropriate be followed by a treatment aiming to regenerate the electrostatic properties of the filter material.

The invention also has for object an installation for carrying out the cleaning method according to the invention.

That installation may include an enclosure in which the filter material to be cleaned, and more particularly the mask or masks, is or are placed and means for injecting into the enclosure the supercritical fluid and any other compound(s), in particular a co-solvent and hydrogen peroxide, in order to expose the filter material to a supercritical fluid, in particular supercritical CO₂, with a mass per unit volume less than 0.3 g/mL.

The invention further has for object a reusable mask cleaned by the cleaning method according to the invention. That mask may be repackaged individually or with others, where necessary.

The cleaned mask may carry an identifier enabling access to a database in which the number of cleaning cycles is stored, with other information. The mask may further carry an indication, for example printed on it or in the form of a label stuck to it, that the mask has undergone cleaning, and even indicating the number of cleaning cycles to which it has been subjected.

The invention further has for object a reusable respiratory protection mask, in particular one including a non-woven fabric, preferably of meltblown type, in particular a mask of FFP2 or FFP3 type, including a colored indicator adapted to become progressively discolored on each cleaning cycle using the supercritical fluid and to reach a predefined degree of discoloration when the mask is no longer conform. The indicator may be produced so that it loses its color before the filter material no longer conforms. Alternatively, the indicator retains a little of its color at the moment the mask is no longer conform, but compared to a reference scale, it is possible to determine if the mask is still conform or not.

The invention further has for object a reusable respiratory protection mask, in particular one including a non-woven fabric, preferably of meltblown type, in particular a mask of FFP2 or FFP3 type, including an identifier making it possible to assure traceability of its cleaning by the cleaning method according to the invention.

In the present application by “mask of FFP2 or FFP3 type” is meant so-called “Covid” masks conforming to the standards NF EN 149: 2001+A1: 2009 relating to FFP2 and FFP3 masks, as well as their foreign equivalents, namely those conforming to the American standard NIOSH 42 CFR 84/N95 as well as P95 and R95, the Chinese standard GB2626-2006/KN95 and KP95, GB/T 32610-2016/class A, the Australian and New Zealand standard AS/NZS 1716:2012/P2, the Korean standard KMOEL-2017-64/1^{st} class, the Japanese standard Japan JMHL W-Notification 214, 2018/DS2 and DL2, the Brazilian standard ABNT/NBR 13698:2011/PFF2, the Mexican standard NOM-116-2009/N95 and P95, R85, the American standard NIOSH 42 CFR 84/N99 and N100, P99, P100, R99, R100, the Chinese standard GB2626-2006/KN100 and KP100, the Australian and New Zealand standard AS/NZS 1716:2012/P3, the Japanese standard Japan JMHLW-Notification 214, 2018/DS3 and DL3, the Brazilian standard ABNT/NBR 13698:2011/PFF3 and the Mexican standard NOM-116-2009/N99 and N100, P99, P100, R99 and R100.

### BRIEF DESCRIPTION OF THE DRAWINGS

The invention may be better understood upon reading the following detailed description of non-limiting embodiments thereof and examining the appended drawings, in which:

FIG. 1 is a T/P state diagram illustrating conditions of temperature and of pressure suitable for use of the method according to the invention employing a mass per unit volume of 0.15 g/mL,

FIG. 2 represents results of comparative tests using different masses per unit volume of supercritical CO₂,

FIG. 3 represents in a simplified and schematic manner a treatment installation according to the invention,

FIG. 4 represents an example of a reusable mask carrying a colored indicator, and

FIG. 5 represents an example of a reusable mask carrying an identifier.

### DETAILED DESCRIPTION

In accordance with the invention, cleaning by the supercritical fluid is carried out under conditions assuring a mass per unit volume thereof less than 0.3 g/mL and preferably close to 0.15 g/mL. The supercritical fluid is preferably supercritical CO₂.

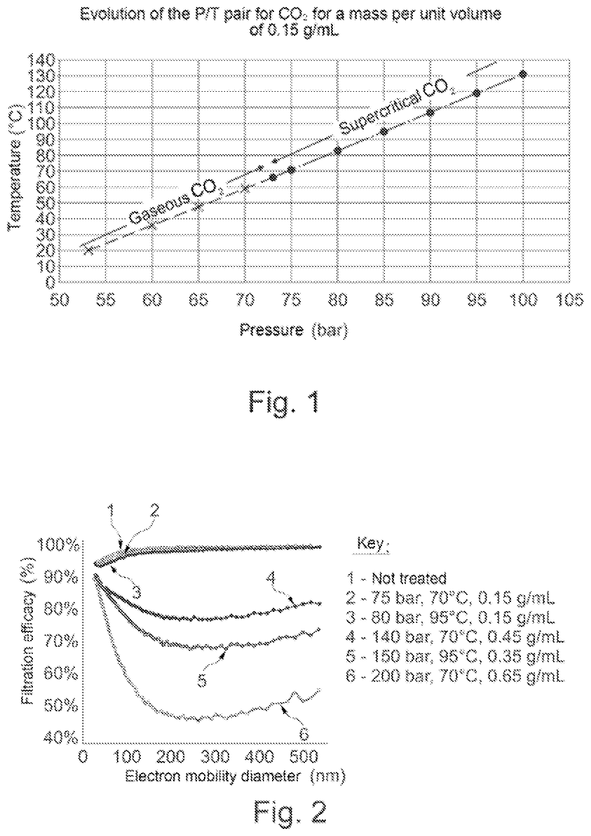

There has been represented in FIG. 1 a diagram showing the evolution of the T/P pair for obtaining supercritical CO₂ with a mass per unit volume equal to 0.15 g/mL.

It is seen that different temperature and pressure pairs may be used to obtain this mass per unit volume of 0.15 g/mL, for example 75 bar and 70° C. or 80 bar and 95° C. Under these conditions the filtration properties of masks of FFP2 or FFP3 type are preserved, in particular those of masks including a polypropylene fiber meltblown type non-woven fabric.

There have been represented in FIG. 2 results of comparative tests showing that for masses per unit volume greater than or equal to 0.35 g/mL this is no longer the case. The curves represent the filtration efficacy as a function of the electron mobility diameter in millimeters. In these tests the masks are treated for one hour with supercritical CO₂ and the rate of expansion at the end of the treatment is 70 bar/min. The masks that were the subject matter of these tests are Kolmi OpAirPro Oxyge FFP2 type masks.

It is seen in FIG. 2 that the filtration efficacy remains substantially the same as that of the original mask for the masks subjected to supercritical CO₂ with a mass per unit volume equal to 0.15 g/mL, whereas this is not the case in the tests in which the masks were subjected to masses per unit volume of 0.35, 0.45 or 0.65 g/mL.

In fact, for a given temperature, the tests show that the filtration efficacy decreases the higher the pressure and thus the mass per unit volume and that the filtration properties are preserved if the pressure is chosen so as to obtain supercritical CO₂ with a mass per unit volume equal to 0.15 g/mL (see tests at 70° C. and 75/140/200 bar and tests at 95° C. and 80/150 bar).

Thus the invention enables the reuse of treated masks of FFP2 or FFP3 type. In fact, the invention makes it possible to clean the masks whilst preserving their filtration properties, that is to say preserving both their mechanical barrier, i.e. the fibrous structure, and their electrostatic barrier, i.e. the presence of surface electrical charge.

To reinforce the biocidal character of the treatment, at least one biocidal compound, for example oxygenated water, is advantageously added to the supercritical CO₂.

Given that water is weakly soluble in supercritical CO₂ an organic co-solvent, for example ethanol, having an affinity for water and CO₂ is added.

Thus in embodiments of the invention the supercritical CO₂ is mixed with alcohol and with oxygenated water at 30 wt % in water in the following proportions:
Supercritical CO₂: 300 mL
Oxygenated water 30 wt % in water: 0.25 mL
Ethanol: 0.25 mL

Tests have shown that treatment using a mixture of this kind had a biocidal activity, including against highly-resistant germs such as spores of Geobacillus stearothermophilus.

Likewise, tests with blood spots of animal origin on the masks show that the method has good cleaning action for this kind of soiling.

The inventors have found that carrying out expansion at a certain rate, not too low, nor too high, leads to the best results where the removal of soiling is concerned, whilst avoiding the use of any mechanical agitation, as emerges from table 1 below.

TABLE 1
Comparison of the effects of treatment with supercritical CO₂ on
agitation in the reactor and cleaning of soiling on masks (visual
observation) as a function of the treatment rate: ±, low effectiveness; +,
moderate effectiveness; +++ very effectiveness/performance

| | Agitation in the reactor | Cleaning of soiling |
|---|---|---|
| Slow expansion | + | ± |
| ~30 bar.min⁻¹ | | |
| Fast expansion | +++ | +++ |
| ~70 bar.min⁻¹ | | |

It is seen in table 1 that the expansion rate is therefore preferably between 60 and 80 bar/min inclusive. An expansion rate of this kind makes it possible to create agitation and therefore significantly to improve the removal of soiling. It is therefore possible not to use mechanical agitation, for example by rotation of the treatment enclosure, since it is the expansion that creates the agitation.

The expansion is preferably effected with at least one stage at substantially P₀/2, P₀ designating the pressure during treatment. The P₀/2 stage may in itself be relatively short, for example of the order of a few seconds.

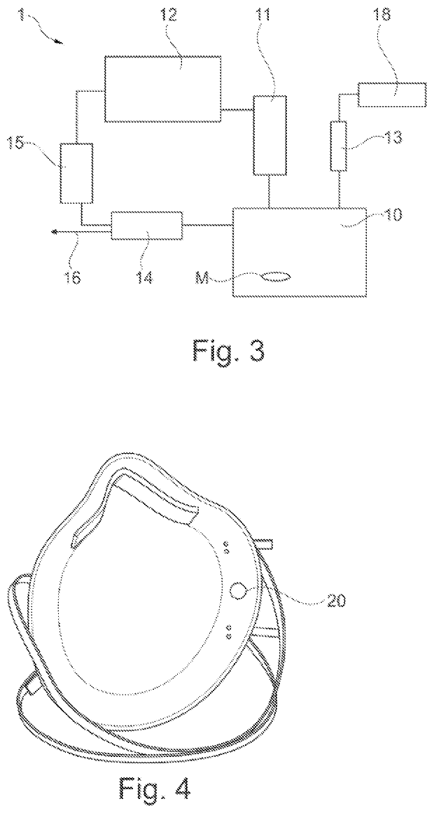

The method may be carried out in an installation 1 as represented schematically in FIG. 3.

The installation 1 includes a treatment enclosure 10, for example of the autoclave type, in which the mask or masks M to be treated is or are placed.

Supercritical CO₂ may be injected by means of a pump 11 connected to a reservoir 12 storing CO₂.

Additional compounds contained in at least one reservoir 18 may be introduced into the enclosure 10 by any appropriate means, for example by means of a pump 13.

The installation advantageously includes a programmable pressure and temperature regulator enabling a predefined temperature and pressure cycle to be tracked.

The supercritical CO₂ is expanded via a separator 14 enabling recovery of the CO₂ without the additional compounds, which may be contaminated, and which may be evacuated at 16 to any appropriate recovery means.

The gaseous CO₂ at the outlet of the separator 14 may be fed into a filter 15, for example an activated carbon filter, before being stored in the reservoir 12 for subsequent reuse.

The enclosure 10 may have no mechanical agitator and the masks M may remain immobile inside it during the treatment.

Tracking of the number of cleaning cycles that the same mask has undergone is preferably provided.

One way of providing this tracking is to provide the mask with a colored indicator 20 as illustrated in FIG. 4, produced so as to lose only part of its color on each cleaning cycle. Thus the choice may be made to produce this mark using an ink the solubility of which in supercritical CO₂ is known and enables it to resist a little on each cleaning cycle.

The mark may be produced so that its erasure substantially coincides with the maximum number of washing cycles that is authorized for the mask.

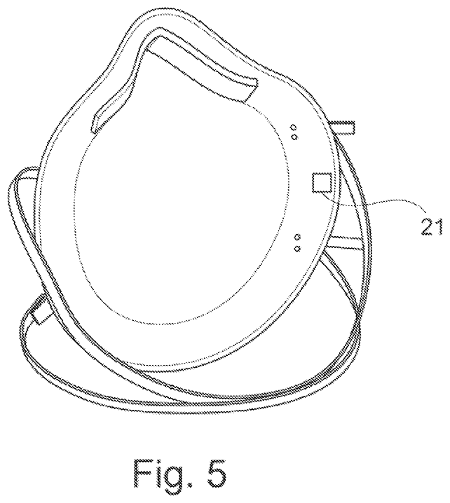

As illustrated in FIG. 5 the mask may likewise be provided with an identifier 21, for example a bar code or QR code, or an RFID chip, for supplying a database with information as to the number of cleaning cycles to which the mask has been subjected and thus to be sure that a given mask has not undergone too high a number of cleanings.

The presence of an identifier may if appropriate enable the cleaned mask to be redistributed to the same user.

If a plurality of masks are repackaged together, the repackaged masks have preferably undergone the same number of cleaning cycles.

Of course, the invention is not limited to the examples that have just been described.

For example, although the invention applies in particular to masks of FFP2 or FFP3 type, it may equally be applied to surgical type masks including a meltblown type non-woven fabric, in particular one based on polypropylene fibers, for example Spunbond-Meltblown-Spunbond (SMS) surgical masks.

The treated masks may be equipped with valves or not.

The invention applies equally to cleaning filter materials in disk form, for example, intended to be removably mounted in corresponding housings on shells of plastic material masks.

The supercritical fluid may be other than supercritical CO₂, for example nitrogen protoxide N₂O which has similar characteristics, or a mixture of CO₂ and N₂O.

If necessary, the masks or the filter material may after cleaning be subjected to any additional treatment aiming to reinforce its filter properties.

If necessary, other compounds, in particular biocides, may be added to the supercritical fluid.

The cleaned masks may be repackaged, in particular in a plastic material sachet or cardboard box.

1.-18. (canceled)

19. A method of cleaning at least one filter material comprising subjecting the filter material to a supercritical fluid of a mass per unit volume less than 0.3 g/mL.

20. The method as claimed in claim 19, wherein the supercritical fluid is supercritical CO₂.

21. The method as claimed in claim 19, wherein the supercritical fluid has a mass per unit volume less than or equal to 0.2.

22. The method of claim 21, wherein the supercritical fluid has a mass per unit volume less than or equal to 0.16 g/mL.

23. The method as claimed in claim 19, wherein the temperature of the supercritical fluid is less than or equal to 130° C.

24. The method of claim 23, wherein the temperature of the supercritical fluid is between 50° C. and 130° C. inclusive.

25. The method of claim 23, wherein the temperature of the supercritical fluid is between 70° C. and 100° C. inclusive.

26. The method as claimed in claim 19, wherein the pressure of the supercritical fluid is between 75 and 100 bar inclusive.

27. The method as claimed in claim 19, wherein the filter material is exposed to a mixture of the supercritical fluid and at least one additional biocidal compound.

28. The method as claimed in claim 27, wherein the total content of the additional biocidal compounds is less than or equal to 2 mole % relative to the total number of moles in the mixture.

29. The method as claimed in claim 27, wherein the additional biocidal compounds are chosen from oxidants and acids and mixtures thereof.

30. The method as claimed in claim 27, wherein the mixture includes water and at least one organic co-solvent chosen from carbon chain alcohols with three carbon atoms or fewer, carboxylic acids with three carbon atoms of or fewer, solutions of polyethylene glycol (PEG) having a molecular mass less than 200 g·mol⁻¹, acetone, dimethylsulfoxide (DMSO), and mixtures thereof.

31. The method as claim in claim 30, wherein the total content of co-solvent(s) is less than or equal to 1.5 mole % relative to the total number of moles in the mixture.

32. The method as claimed in claim 19, wherein the filter material is exposed to the supercritical fluid without mechanical agitation produced by stirring, by ultrasound or by imparting movement to the filter material.

33. The method as claimed in claim 19, wherein the filter material is exposed to expansion of the supercritical fluid at an expansion rate between 60 bar/min et 80 bar/min inclusive.

34. The method of claim 33, wherein the filter material is exposed to expansion of the supercritical fluid with at least one decompression stage.

35. The method as claimed in claim 19, including a step of counting the number of cleaning cycles to which the filter material has been subjected.

36. The method as claimed in claim 19, wherein the filter material or the mask which is made of it carrying a visual indicator the color of which pales as the cleaning cycles proceed, the method including a step of verifying that the indicator is colored to more than a certain degree before allowing reuse of the filter material.

37. The method as claimed in claim 19, applied to the cleaning of masks of FFP2 or FFP3 type.

38. The method as claimed in claim 19, applied to cleaning polypropylene fiber-based meltblown type non-woven fabric.

39. An installation for the execution of the method as defined in claim 1, including an enclosure in which the filter material or the mask or masks to be cleaned is or are placed and means for injecting into the enclosure the supercritical fluid and any other compound(s) in order to expose the filter material or the masks to a supercritical fluid having a mass per unit volume less than 0.3 g/mL.

40. A respiratory protection mask reusable after cleaning, including a non-woven fabric, including a colored indicator adapted to become progressively discolored in each cleaning cycle using the supercritical fluid, in particular the supercritical CO₂, during use of the method as defined in claim 1, and to reach a predefined degree of discoloration when the mask is no longer conform.

41. A respiratory protection mask reusable after cleaning, including a non-woven fabric, and an identifier enabling tracking of the cleaning thereof using the method as claimed in claim 1.
